(19) United States
(12) Patent Application Publication
(10) Pub. No.: US 2012/0064533 A1
(43) Pub. Date: Mar. 15, 2012

(54) ASSAY FOR DETECTING VITAMIN D AND ANTIBODIES THEREFOR

(76) Inventors: Margaret Lawlor, Dundee (GB); David Pritchard, Perthshire (GB); Murdo Black, Forfar (GB); Lars Ørning, Oslo (NO)

(21) Appl. No.: 13/254,818

(22) PCT Filed: Mar. 2, 2010
(86) PCT No.: PCT/GB2010/050368

§ 371 Date: Nov. 29, 2011

(30) Foreign Application Priority Data
Mar. 2, 2009 (GB) 0903469.5

(51) Int. Cl. C12Q 1/68; G01N 33/82; C07K 1/14; C07K 16/18

(52) U.S. Cl. 435/6.12; 530/389.1; 436/501; 530/388.2; 530/391.3; 530/392; 435/7.92

(57) ABSTRACT

A molecule which recognises holo-DBP but which does not recognise apo-DBP or has relatively low affinity thereto.

The present invention relates to assaying for vitamin D and in particular to assaying for vitamin D in complex with vitamin D binding protein.

In animals, Vitamin D principally operates to maintain the circulating levels of calcium and phosphate in the blood, substances which in vivo affect properties/processes such as bone mineralisation, muscle contraction and nerve conduction as well as other general cellular functions. As such, variation in vitamin D concentration can impact on muscle function and the immune, nervous, heart and circulatory systems, as well as being linked to diverse medical conditions such as bone disease, type II diabetes and cancer.

Vitamin D is found in various forms, the principal two of which are considered to be D₂ and D₃, though the term is recognised by those skilled in the art to refer broadly to the family of related fat soluble molecules as well as metabolites, derivatives and other analogues of these substances. Accordingly, hereinafter vitamin D will be used to refer to all the members of the group of molecules collectively known and/or broadly described/referred to as vitamin D in the art.

Vitamin D can be acquired in the diet or produced photochemically in the skin through the action of sunlight. Vitamin D₂ is not known to be synthesised by animals and is acquired in the diet from fungal and plant sources. Vitamin D3 can similarly be acquired by animals via a carnivorous diet or, as said above, can be synthesised de novo in the skin. Regardless of its source, however, vitamin D is principally stored in vivo in the 25 hydroxyvitamin D (hereinafter 25(OH)D) form, which is produced by hydroxylation of vitamin D in the liver. 25(OH)D is the major form of vitamin D found in blood and is the precursor of 1, dihydroxyvitamin D (hereinafter 1.25(OH)₂D), a physiologically active form of vitamin D produced mainly in the kidney. 1.25(OH)₂D binds to the vitamin D receptor (VDR) which, upon said binding, can subsequently mediate both rapid and long-term genomic responses.

The biologically active forms of vitamin D function by binding to the vitamin D receptor (VDR) in target cells, for example in the intestine, bone or kidney. Vitamin D is poorly soluble in water and so, to enable its transport in the blood to its target cells, it is bound in complex with a soluble 52 kDa carrier protein known as vitamin D binding protein or group specific component (Gc). The vitamin D binding protein has variants including but not restricted to type 1S, 1F and 2. Hereinafter the group of proteins including all variants known in the art as vitamin D binding protein or Gc will be referred to as DBP.

DBP concentration is ordinarily 20-times that of vitamin D in the blood and so, since DBP has very high binding affinity for vitamin D, most blood vitamin D is found bound to DBP. The vitamin D-DBP complex is hereinafter referred to as holo-DBP.

In terms of binding activity, DBP binds to forms/metabolites of vitamin D with the following relative affinities: 25 hydroxyvitamin D (25(OH)D)=24, 25 dihydroxyvitamin D (24, 25(OH)₂D)>1.25 dihydroxyvitamin D (1, 25(OH)₂D)>cholcalciferol.

Of circulating 25 (OH) D, more than 90% is normally found bound to DBP and though other proteins, such as human serum albumin (HSA,) have been shown to bind 25(OH)D, it is with significantly lower affinity.

Given its wide-reaching and substantial impact on health, there is an obvious medical requirement to be able to easily and accurately measure in vivo vitamin D levels.

There are two main forms of vitamin D which can be measured in bodily fluids, 25(OH)D and 1.25(OH)₂D. As said above, 25(OH)D is normally found at a higher concentration in the blood than 1.25(OH)₂D, and, since it also has a relatively long half-life, 25(OH)D is commonly measured to assess and monitor vitamin D status in individuals. Furthermore, being the active form of vitamin D, the plasma/serum levels of 1.25(OH)₂D tend to be maintained at a constant level, so 25(OH)D levels are a better indicator of an individual's overall circulating vitamin D status. Determining levels of 1.25(OH)₂D, however, serves as an indication of whether there is adequate production in the kidney(s) of the active form of the compound.

It is common practice when seeking to determine the levels of a target to measure it directly. Accordingly, current assays to determine vitamin D levels require that vitamin D is initially dissociated from its DBPs before measuring the level of vitamin D, which is poorly soluble in aqueous media. This separation may be achieved by denaturing and removing DBPs or using a displacement reagent to release vitamin D and then measuring the resultant free vitamin D. The requirement for dissociation of the DBPs obviously necessitates incorporation of such a step in any vitamin D assay and so adds to the time and complexity of conducting the assay.

Due to the hydrophobic nature of vitamin D, a current assay technique, for example, is to use solvents to release the vitamin D and denature the DBP. Such an approach necessitates subsequent separation of the denatured DBP and solvent extraction before vitamin D content can be assessed.

Furthermore, current methods of assessing vitamin D levels are reported to suffer from significant variability problems, in relation to both individual techniques and also when different assays are compared. As process complexity increases, so issues such as reliability become more of a challenge. Levels of imprecision become very important with high sensitivity assays, and because of the multi-stage nature of existing processes (including vitamin D displacement, liquid additions and removals, and multiple wash steps), errors can accumulate and precision suffers accordingly. Consequently, there is a need for a simplified vitamin D assay which is likely to provide lower imprecision.

To address these issues, the present invention seeks to overcome problems associated with the prior art.

According to a first aspect of the present invention, there is provided a molecule which recognises holo-DBP but which does not recognise apo-DBP (also known as unbound DBP or DBP) or has a relatively low affinity thereto. Relatively low affinity may be, for example, represented by the molecule exhibiting cross-reactivity to apo-DBP of <10% or preferably <1%, such that it binds preferentially to holo-DBP over apo-DBP.

This recognition molecule will accurately and effectively be able to distinguish between holo-DBP and apo-DBP. This will allow for the use of this molecule in a test for the detection of vitamin D in samples, such as tissue samples or bodily fluids. In this way, by permitting selective determination of the holo and apo forms of DBP the present invention allows for more precise, rapid and beneficially targeted measurement of vitamin D levels than is granted by existing procedures.

The recognition molecule could primarily recognise vitamin D but crossreact with holoDBP such that holoDBP is recognised. In this connection, use of the word “recognition” (or any derivative thereof (e.g. recognise)) in this specification is considered to cover primary recognition, cross-reactivity or any secondary or subsequent form of recognition. The term ‘recognition molecule’ will be understood to encompass molecules that interact with or bind to a target in any way.

Preferably, said recognition molecule may be used to capture, concentrate, separate or detect holo-DBP.

Preferably, the recognition molecule does not recognise unbound vitamin D. Alternatively, the recognition molecule can recognise unbound vitamin D along with holo-DBP in the sample.

Preferably, the recognition molecule will exhibit selectivity towards a particular variant form(s) of vitamin D, for example 25(OH)D.

Preferably, the DBP used for generating and testing the recognition molecule is mixed-type DBP, i.e. DBP that contains a mixture of genetic variants of the protein. Alternatively, the DBP may be a specific genetic variant of DBP.

Conveniently, said recognition molecule is selected from an antibody (e.g. a monoclonal antibody), antibody fragment (e.g. F(ab), F(ab′)2, F(v), scFv), protein, molecular imprinted polymer (MIP), a complementarity determining region (CDR), oligopeptide, oligonucleotide (e.g. an aptamer) or small organic chemical which has affinity for holo-DBP.

Optionally, said recognition molecule comprises a signal component, wherein said component is a radioisotope, fluorophore, chromophore, binding ligand or enzyme substrate, such that said signal component enables detection of said composition. Examples of signal molecules which can be conjugated to the recognition molecule are, but are not limited to, enzymes such as horseradish peroxidase, malate dehydrogenase, staphylococcal nuclease, delta-5-steroid isomerase, yeast alcohol dehydrogenase, alpha-glycerophosphate dehydrogenase, triose phosphate isomerase, alkaline phosphatase, asparaginase, glucose oxidase, beta-galactosidase, ribonuclease, urease, catalase, glucose-6-phosphate dehydrogenase, glucoamylase, and acetylcholine esterase, chemiluminescent compounds such as acridinium ester, suitable fluorescent labelling compounds include fluorescein isothiocyanate, rhodamine, phycoerythrin, phycocyanin, allophycocyanin, o-phthaldehyde and fluorescamine.

According to a further aspect of the present invention there is provided a molecule that competes with a recognition molecule as described herein.

According to a further aspect of the present invention there is provided a method for the detection of vitamin D in a sample, the method comprising contacting the sample with the molecule of the present invention and detecting the amount of vitamin D present in the sample.

This method negates the requirement for dissociation of vitamin D from DBPs when determining vitamin D levels, and so allows for more rapid and simpler assaying of vitamin D. The amount of holo-DBP in the sample will have a direct relationship with the amount of vitamin D.

The method could also be used to detect different forms of vitamin D depending upon the recognition molecule used. For example, if the function of the kidneys in processing vitamin D was to be assessed then a recognition molecule for 1.25(OH)₂D could be used, or if the amount of vitamin D in the blood was to be assessed then either a general recognition molecule which recognises all vitamin D bound to DBP could be used or one which recognises the major form of vitamin D in the blood, 25(OH)D, bound to DBP could be used.

Conveniently, the sample can be any tissue sample or bodily fluid, preferably, blood, serum or plasma.

It will be understood by those skilled in the art that such a method could be performed in a great variety of different ways and relying on different principles. For example, in use the recognition molecule may be, or may be able to be, immobilised to enable separation of holo-DBP from the remainder of a sample. If said recognition molecule is immobilised it may be coupled either directly or indirectly to a solid surface such as a filter or wall of a tube or as a further example to a support or matrix (which may be loaded in a column) for use in liquid chromatography. If the recognition molecule is able to be immobilised, this may be achieved by coupling the molecule to a member of a specific binding partnership (for example biotin/streptavidin) and subsequently using another member of the partnership to immobilise or agglomerate the molecule from the remainder of a sample.

Those skilled in the art will further understand that the method may possibly be performed as a competitive or displacement assay, whereby, for example, a molecule, which may optionally be labelled, that competes with holo-DBP for binding to the recognition molecule (i.e. a holo-DBP competing molecule) may be used. Further, it will be understood that additional molecules which interact with the holo-DBP recognition molecule and/or the holo-DBP-recognition molecule complex may also be used in certain embodiments of the invention.

In varying embodiments of the present invention it may be desirable to measure the recognition molecule and/or the holo-DBP competing molecule, either or both of which may be immobilised, in solution or precipitated. In a further embodiment it is convenient to measure the spatial interaction between appropriately labelled holo-DBP competing molecule and appropriately labelled recognition molecule.

There is a wide range of detection techniques which can be exploited in different embodiments of the present invention. Direct assessment such as mass spectrometry or HPLC could be used or, conveniently, a component used may be coupled to a signal molecule or label, for example one which is luminescent, chemiluminescent, radioactive, fluorescent, suitable for colorimetric detection, a binding protein, an epitope or an enzyme or substrate. Further, a radio-label or electrochemical label, may optionally be incorporated into a component. In practice, any signal molecule or label known in the art may possibly be incorporated in embodiments of the present invention.

Persons skilled in the art will appreciate that the method of the current invention can be performed using a variety of systems, for example radioimmunometric assays (IRMA) and enzyme linked immunosorbent assays (ELISA), microparticle enzyme immunoassays (MEIA), immunoprecipitation and liquid chromatography, as well as direct assay formats such as Surface Plasmon Resonance, Surface Acoustic Wave and Quartz Crystal Microbalance methodologies.

Conveniently, the recognition molecule is immobilised on a microtitre plate or on a microparticle such as a bead wherein the bead comprises latex, polystyrene, silica, chelating sepharose and/or is magnetic.

According to a further aspect of the present invention there is provided a method for separating holo-DBP from apo-DBP using a recognition molecule as described herein.

According to a further aspect of the present invention there is provided a kit comprising a molecule as described herein for a method of detecting vitamin D or a method of separating holo-DBP from apo-DBP as described above.

The present invention will now be illustrated by the following non-limiting examples. In the following examples the holo-DBP is a complex of 25-OH-D₃ (obtained from Merck Chemicals Ltd., catalogue number 679102) and mixed type DBP (also obtained from Merck Chemicals Ltd., catalogue number 345802) but could equally relate to a complex of any form of vitamin D and any form of DBP, a specific form of vitamin D and an unspecified form of DBP or an unspecified form of vitamin D and a specific form of DBP.

The invention will be described with reference to the accompanying drawings, in which:

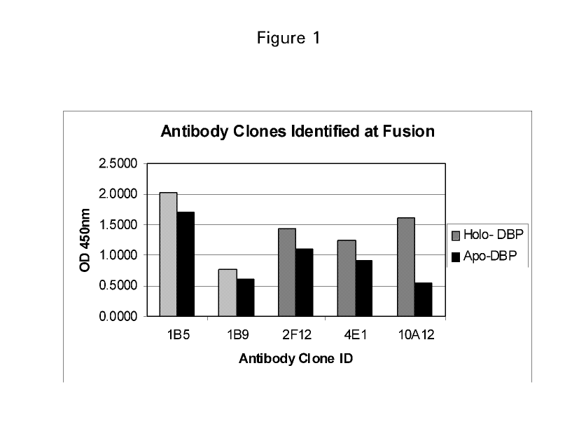

FIG. 1 shows differential binding of holo-DBP antibodies to holo-DBP (1 μg/mL) and apo-DBP (1 μg/mL) after initial fusion of antibody producing spleen cell with the myeloma cells.

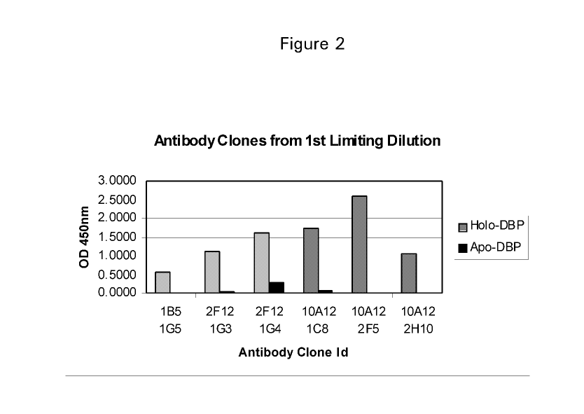

FIG. 2 shows differential binding of holo-DBP antibodies to holo-DBP (1 μg/mL) and apo-DBP (1 μg/mL) after a first round of limiting dilution.

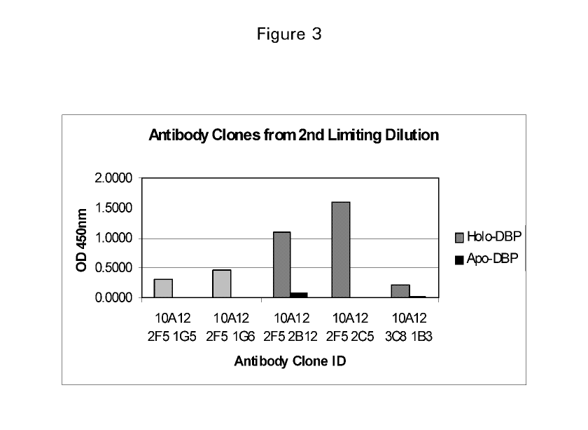

FIG. 3 shows differential binding of holo-DBP antibodies to holo-DBP (1 μg/mL) and apo-DBP (1 μg/mL) after a second round of limiting dilution.

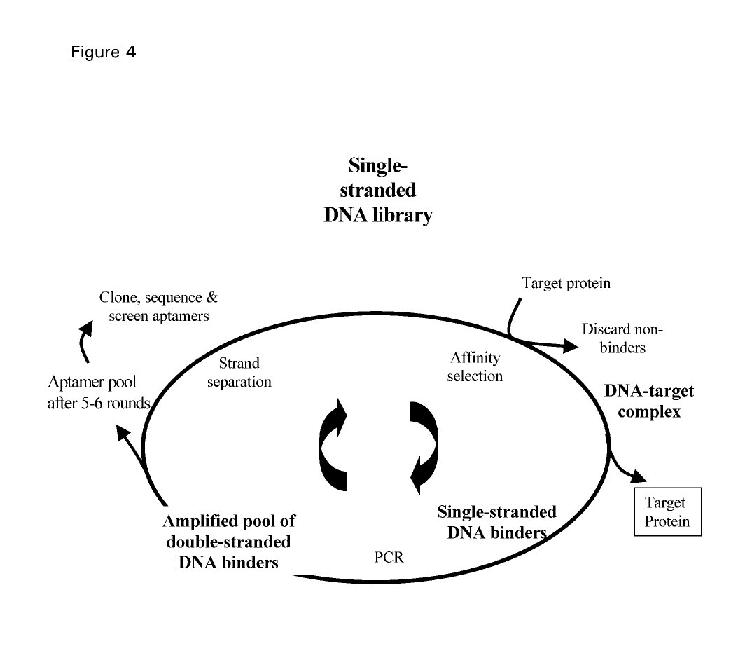

FIG. 4 shows the SELEX process.

### EXAMPLE 1

Preparation of Monoclonal Antibodies

Murine monoclonal antibodies to holo-DBP (Vitamin D complexed to DBP) are prepared by a modification of the method of Kohler and Milstein (G. Kohler and C. Milstein Nature, 1975 256, 495). Female BaLb/C mice are immunised by subcutaneous injection of unconjugated holo-DBP (50 μg per mouse). The immunogen is presented in complete Freund's adjuvant. Antigen boosts presented in an incomplete Freund's adjuvant are given to enhance antibody response (20 μg per mouse per boost). The immune response is monitored by solid phase immunoassay to holo-DBP. The mice are given four or five boosts prior to harvest of the spleen cells and fusion with murine myeloma cells.

Fusion of spleen lymphocytes and myeloma cells

Immune mouse spleen cells are fused with NSO mouse myeloma cells in the presence of PEG. The cells are seeded in wells of culture dishes and grown in the presence of selective HAT (hypoxanthine, aminopterin and thymidine) medium which allows for selection of fused spleen and myeloma cells. Supernatants from the fused cells are tested for reactivity to holo-DBP and/or apo-DBP by standard solid phase immunoassay techniques (see subsequent example(s)).

Isolation of Immunoglobulin fraction from Ascites Fluid

To isolate the immunoglobulin fraction from ascites fluid the immunoglobulin fraction is first precipitated from the ascites fluid at 4° C. by adding an equal volume of saturated ammonium sulphate solution. The precipitate is centrifuged and then dissolved in a volume of standard Tris buffer equal to that of the original ascites fluid and then dialysed against the same Tris buffer. The immunoglobulin solution is further purified using a mono Q column and a salt gradient to elute the immunoglobulins. The immunoglobulin fraction is then tested for immune response to the antigen.

### EXAMPLE 2

Preparation of Polyclonal Antisera

Polyclonal antisera to holo-DBP can be raised in sheep or rabbits using holo-DBP according to the method described in Methods in Enzymology, H. Van Vunatis and J. J. Langone (Eds) 1981, (729b) and 1983, 92(E). Other species known in the art may also be used to generate polyclonal antibodies.

Polyclonal antibodies that are selective for holo-DBP can be isolated using techniques, familiar to those skilled in the art, such as affinity purification. For example, the polyclonal anti-sera can be incubated with a solid phase coated with holo-DBP, after which material that is not bound to the solid phase can be washed away leaving only molecules that can bind to holo-DBP, which can themselves be released from the solid phase by a number of techniques including the use of chaotropic ions. The released antibodies can then be incubated with a solid phase coated with apo-DBP, thus leaving in solution only those antibodies that exhibit specificity for holo-DBP over apo-DBP.

### EXAMPLE 3

Panning of Phage Display Library

Screening for an antibody with the aforementioned properties of binding Vitamin D complexed to DBP but not recognising Vitamin D or DBP alone is performed using combinatorial immunoglobulin libraries on the surface of phage, as described in Barbas C. F. and Lerner, R. A. (1991) Combinatorial immunoglobulin libraries on the surface of phage (phabs). Rapid selection of antigen-specific Fabs. METHODS: A Companion to Methods in Enzymology 2: 119-124.

Phage which display the desired antibodies are selected by “phage panning”, which is a technique with similarities to solid phase immunoassays. The holo-DBP is immobilised on a solid surface such as a microtitre plate, beads such as, but not limited to, magnetic beads, or a column. Phage are then added and allowed to interact with the holo-DBP. After washing to remove all non interacting material, the bound phage are eluted and amplified by replication in new bacterial host cells. To enhance the selection of antibodies for the holo-DBP, increasing concentrations of apo-DBP are added to the phase solution when interacting with holo-DBP. This selection process is repeated several times to select for a population of phage that express only antibodies that bind the holo-DBP.

The antibody genes are next isolated and inserted into an expression vector to produce soluble antibody fragments, as the antibody gene must be expressed without the phage coat protein. This can be achieved by using standard polymerise chain reaction (PCR) amplification or restriction enzymes in which the Fab genes for light and heavy chains are isolated and cloned into a new vector. The fab fragments are tested for differential binding to either apo or holo -DBP coated onto a plate at 5 μg/ mL in phosphate buffered saline (PBS). The Fabs are assayed at a concentration of either 0.1 μg/mL or 2 μg/mL in PBS.

To produce monoclonal antibodies, the vector-Fab genes are transformed into a new host and isolated as single colonies. Several colonies are isolated and antibody expression is induced and, following release of the monoclonal antibodies by cell lysis, the lysate is tested by enzyme linked immunosorbant assay (ELISA) for the presence of antibodies to holo-DBP.

### EXAMPLE 4

Antibody Binding Assays and Clone Selection

NUNC maxisorb plates are coated overnight with 100 μL per well of either holo or apo-DBP at a concentration of 1 μg/mL in carbonate buffer, pH 9.5. The next day the coating mixture is discarded and the plate blocked with BSA (2 mg/mL in phosphate buffered saline (PBS), pH 7.5) for 60 min at room temperature. The plate is washed once with PBS+0.1% tween 20 (PBST). Sample, i.e. bleed/cell supernatant material, diluted in PBS, is incubated with either the holo or apo-DBP for 60 minutes at room temperature. Apo-DBP (i.e. DBP lacking bound vitamin D) at an increasing excess concentration (i.e. from 10 to 100 fold excess compared to the relevant coating concentration of protein, i.e. 1 μg to 10 μg per sample), may also be added to the antibody solution for a 1 hour pre-incubation prior to incubation in holo-DBP coated wells, in order to drive the selection of antibodies specific for holo-DBP and not apo-DBP. Supernatant is then discarded and the plate washed three times with PBST. Secondary antibody (Goat anti-mouse antibody conjugated to Horse Radish Peroxidase, Bio-Rad catalogue number 170-6516) used at 1:5000 dilution in PBS is incubated with the plate for 60 minutes at room temperature. The plates are then washed three times with PBST before tetramethylbenzidine (TMB) substrate (obtained from Biopanda Diagnostics, catalogue number TMB Solution II) is added. The reaction is stopped by adding 2M H₂SO₄. The sample absorbance/optical density readings at 450 nM are then measured on a spectrophotometer. FIG. 1 shows the typical differential binding of antibodies from one set of experiments produced after initial fusion of antibody producing spleen cells with myeloma cells to holo-DBP and apo-DBP. Table 1 shows the cross reactivity of the holo-DBP antibody supernatants with apo-DBP.

TABLE 1
Cross reactivity of the holo-DBP antibody with
apo-DBP for the clones identified after fusion.

| | Cross Reactivity with apo- |
|---|---|
| Fusion Clones | DBP |
| 1B5 | 84.2% |
| 1B9 | 78.0% |
| 2F12 | 77.0% |
| 4E1 | 73.1% |
| 10A12 | 33.9% |

All wells (samples) showing greater specificity for holo-DBP over apo-DBP are further screened. Samples from all wells that yield antibody producing cells are removed and cloned by at least two rounds of standard limiting dilution cloning procedure. After each round of limiting dilution the clone supernatant is tested as indicated above for expression of antibodies showing greater specificity for holo-DBP over apo DBP.

See FIGS. 2 and 3 for the results from one set of experiments for the differential binding of holo-DBP antibody clones to holo- and apo-DBP after first and second rounds, respectively, of limiting dilution and Tables 2 and 3 for the cross reactivity of the holo-DBP antibody supernatants with apo-DBP (Table 2 relates to the samples of FIG. 2 and Table 3 to FIG. 3).

TABLE 2
Cross reactivity of the holo-DBP antibody with
apo-DBP for first limiting dilution clones.

| 1st Limiting Dilution | Cross Reactivity with apo- |
|---|---|
| Clones | DBP |
| 1B5 1G5 | 5.7% |
| 2F12 1G3 | 3.7% |
| 2F12 1G4 | 17.8% |
| 10A12 1C8 | 5.3% |
| 10A12 2F5 | 0.9% |
| 10A12 2H10 | 3.7% |

TABLE 3
Cross reactivity of the holo-DBP antibody with
apo-DBP for second limiting dilution clones.

| 2nd Limiting Dilution | Cross Reactivity with apo- |
|---|---|
| Clones | DBP |
| 10A12 2F5 1G5 | 7.3% |
| 10A12 2F5 1G6 | 4.4% |
| 10A12 2F5 2B12 | 8.1% |
| 10A12 2F5 2C5 | 1.6% |
| 10A12 3C8 1B3 | 13.8% |

The antibody mRNA may also be isolated from the clone cell pellets. The mRNA is converted to cDNA using a reverse transcriptase. PCR reactions using variable domain primers to amplify both the variable heavy and variable light chain regions of the monoclonal antibody DNA are performed and the variable heavy and light chains (scFv) or the Fab fragments of the antibody (but not limited to these antibody fragments) may be expressed in another expression system such as E. coli, yeast, mammalian cell lines or another expression system known in the art using standard molecular biology techniques. Manipulation of the linker length between variable heavy and variable light chains as described in Le Gall F. et al FEBS Letters, 453 (1999)164-168 and Kortt A. A. et al Biomolecular Engineering,18 (2001) 95-108 can be used to select for expression of of multimeric (for example but not limited to dimeric, trimeric) scFv antibody fragments.

### EXAMPLE 5

Preparation of Aptamers

Isolation of DNA or RNA aptamers is performed using the in vitro selection process Systemic Evolution of Ligands by EXponential enrichment (SELEX). See FIG. 4 for the SELEX process.

A pool of approximately 10¹⁴ oligonucleotide sequences, each with a unique sequence, are generated using standard automated oligonucleotide synthesis methods. Starting pools typically have, but are not limited to, sequence lengths in the range of approximately 60-80 nucleotides.

The oligonucleotides are then screened against immobilised holo-DBP as described in Example 6. The holo-DBP is immobilised on a solid surface such as a microtitre plate, beads such as, but not limited to, magnetic beads, or a column. The oligonucleotides are allowed to incubate with holo-DBP in the presence of increasing amounts of apo-DBP. Molecules with low affinity remain in solution and can be removed by washing. Any holo-DBP bound oligonucleotides, i.e. aptamers, are the purified away from the holo-DBP, amplified and used in subsequent rounds of the SELEX process. The cycle is repeated until aptamers to holo-DBP are the majority of the remaining oligonucleotide population.

Final aptamers usually have lengths in the range of 20-40 nucleotides, these aptamers are cloned and sequenced.

The SELEX Process for Aptamer Generation as set out in FIG. 4 was used.

### EXAMPLE 6

Indirect Assay formats Using Recognition Molecules for holo-DBP

Examples of suitable assay formats for indirect measurement of holo-DBP, and thus the vitamin D levels in plasma/serum samples, include radioimmunometric assays (IRMA) and enzyme linked immunosorbent assays (ELISA) and microparticle enzyme immunoassays (MEIA). In a competitive assay, the holo-DBP can be labelled with a detectable label. The sample containing the holo-DBP can be incubated with the holo-DBP-specific recognition molecule and the labelled holo-DBP, and after formation of immune complexes, separation and detection, the level of holo-DBP in the sample can be determined. The amount of holo-DBP is related to the concentration of vitamin D in the sample.

If the recognition molecule is specific for, for example, 25(OH)D bound to DBP, the concentration of 25(OH)D in the sample will be measured.

Examples of signal molecules which can be conjugated to the recognition molecule are, but are not limited to, enzymes such as horseradish peroxidase, malate dehydrogenase, staphylococcal nuclease, delta-5-steroid isomerase, yeast alcohol dehydrogenase, alpha-glycerophosphate dehydrogenase, triose phosphate isomerase, alkaline phosphatase, asparaginase, glucose oxidase, beta-galactosidase, ribonuclease, urease, catalase, glucose-6-phosphate dehydrogenase, glucoamylase, and acetylcholine esterase, chemiluminescent compounds such as acridinium ester, suitable fluorescent labelling compounds include fluorescein isothiocyanate, rhodamine, phycoerythrin, phycocyanin, allophycocyanin, o-phthaldehyde and fluorescamine.

The recognition molecule may otherwise be radio-labelled or electrochemically labelled.

In brief, the holo-DBP and thus the vitamin D levels in plasma or serum samples are measured by capturing the holo-DBP on a solid surface such as, but not limited to, the surface of a 96-well plate or magnetic or latex microparticle pre-coated with an anti-DBP antibody. Holo-DBP is then measured using the anti holo-DBP antibody conjugated to a signal molecule as described previously. The concentration of vitamin D is determined from a standard curve.

Conversely, the microtitre plate or the microparticles (which may be latex or magnetic) may be coated with the anti holo-DBP antibody or other recognition molecule. This antibody or other recognition molecule captures the holo-DBP in the plasma or serum sample. The microtitre well/microparticle is then washed to remove unbound sample material and a labelled anti-DBP antibody, anti-vitamin D antibody, anti-holo-DBP antibody or other recognition molecule is incubated with the captured holo-DBP. If necessary, substrate is then added and the presence of the label is measured. The amount of signal generated is directly related to the amount of holo-DBP in the sample. The amount of holo-DBP in the sample is directly related to the concentration of vitamin D in the sample and can be measured from a standard curve.

### EXAMPLE 7

Direct Assay Formats Using Recognition Molecules for Holo-DBP

In brief, the holo-DBP recognition molecule may also be used in direct detection of holo-DBP and thus 25 (OH) Vitamin D. For example, using techniques such as Surface Plasmon Resonance, Surface Acoustic Wave and Quartz Crystal Microbalance methodologies (Suzuki M, Ozawa F, Sugimoto W, Aso S. Anal Bioanal Chem 372:301-304 2002; Pearson J E, Kane J W, Petraki-Kallioti I, Gill A, Vadgama P. J Immunol Methods; 221 :81-94 1998; Weisch W, Klein C, von Schickfus M, Hunklinger S. Anal Chem; 68 2000-20004, 1996; Chou S F, Hsu W L, Huang J M, Chen C Y. Clin Chem 48:913-918, 2002).

### EXAMPLE 8

Proximity Ligation Assay

Proximity probing, also termed proximity ligation, is a technique capable of detecting proximity probes and is used for specific, sensitive and rapid detection of macromolecules such as proteins. Proximity ligation relies on two adherent molecules which can be antibodies, peptides, proteins, or aptamers bound to individual non-overlapping synthetic oligonucleotide to be brought into spatial proximity through binding a target molecule such as vitamin D. A third oligonucleotide is introduced that acts as a bridge to bring the two non-overlapping oligonucleotides together, allowing a DNA ligase to complete a contiguous DNA element. Real time fluorometric polymerase chain reaction allows amplification of only the DNA fragments that have been successfully ligated together.

1. A molecule which recognises vitamin D complexed to vitamin D binding protein (holo-DBP1) but which does not recognise unbound vitamin D binding protein (apo-DBP) or has a lower affinity for apo-DBP than for holo-DBP.

2. A molecule according to claim 1, wherein the molecule has a lower affinity for apo-DBP than for holo-DBP as evidenced by a cross-reactivity to apo-DBP is of less than 10%.

3. A molecule according to claim 2, wherein cross-reactivity to apo-DBP is less than 1%.

4. A molecule according to claim 1, which further comprises a signal component.

5. A molecule according to claim 1, wherein the molecule does not recognise vitamin D.

6. A molecule according to claim 1, wherein the molecule recognises vitamin D.

7. A molecule according to claim 1, wherein the holo-DBP comprises 25-hydroxyl-vitamin D (25(OH)D) associated with vitamin D binding protein (DBP).

8. A molecule according to claim 1, wherein the DBP is a mixed type DBP.

9. A molecule according to claim 1, wherein the molecule is an antibody or a fragment thereof.

10. A method for the detection of vitamin D in a sample, the method comprising:
contacting the sample with a molecule according to claim 1, and
determining the amount of vitamin D present in the sample.

11. A method according to claim 10, wherein an amount of bound holo-DBP is indicative of the amount of vitamin D in the sample.

12. A method according to claim 10, wherein the sample is a bodily fluid.

13. A method according to claim 12, wherein the bodily fluid is plasma or serum.

14. A method for separating holo-DBP from apo-DBP, comprising:
contacting a sample with the molecule according to claim 1.

15. A kit, comprising: a molecule according to claim 1.

16. The kit according to claim 15, wherein the molecule is an antibody or a fragment thereof.

17. The kit according to claim 15, wherein the molecule is a monoclonal antibody or a polyclonal antibody.

18. A molecule according to claim 9, wherein the molecule is an antibody comprising a signal component.

19. A method according to claim 10, wherein the molecule is a monoclonal antibody or a polyclonal antibody.

20. A method according to claim 19, wherein the molecule further comprises a signal component.
